(19) United States
(12) Patent Application Publication
(10) Pub. No.: US 2012/0316428 A1
(43) Pub. Date: Dec. 13, 2012

(54) ARRANGEMENT FOR EXAMINATION AND/OR TREATMENT OF A HUMAN OR ANIMAL BODY

(76) Inventors: Felix Güttler, Berlin (DE); Jens Rump, Berlin (DE); Ulf Teichgraber, Berlin (DE)

(21) Appl. No.: 13/513,143

(22) PCT Filed: Dec. 15, 2010
(86) PCT No.: PCT/DE10/50090

§ 371 Date: Aug. 14, 2012

(30) Foreign Application Priority Data
Dec. 17, 2009 (DE) 10 2009 060 109.0

(51) Int. Cl. A61B 5/055

(52) U.S. Cl. 600/418

(57) ABSTRACT

The invention relates to an arrangement (10) for examination and/or treatment of a human or animal body having a shielded room (20), a magnetic resonance imaging device (MRI) which is arranged within the shielded room, and at least one communication device (30), which is arranged within the shielded room and is suitable for receiving a radio signal for transmission of a voice signal, and has at least one electro-acoustic transducer (40, 41) which is suitable for conversion of an audio signal, which has been transmitted by the radio signal (Fin), to an acoustic signal (EAA). The invention provides that at least one antenna, which is connected to at least one further communication device, is arranged in the shielded room and is connected by radio to the at least one communication device (30) which is arranged within the shielded room.

The invention relates to an arrangement for examination and/or treatment of a human or animal body, having the features according to the precharacterizing clause of patent claim 1.

An arrangement such as this is known from German Laid-Open Specification DE 103 43 006. This document describes an electro-acoustic system for use in the interior of a magnetic-field resonance imaging device having a loudspeaker with an electrodynamically excited oscillating system for sound production, which is designed taking account of the static basic magnetic field in the magnetic-field resonance imaging device. The oscillating system comprises an oscillating coil, whose axis is arranged parallel to the basic magnetic field.

The invention is based on the object of specifying an arrangement for examination and/or treatment of a human or animal body, in which interaction between the personnel can be carried out more easily and better than in the case of already known arrangements.

According to the invention, this object is achieved by an arrangement having the features according to patent claim 1. Advantageous refinements of the arrangement according to the invention are specified in dependent claims.

According to the invention, at least one antenna, which is connected to at least one further communication device, is arranged in the shielded room and is connected by radio, preferably a voice radio link, to the at least one communication device which is arranged within the shielded room.

One major advantage of the arrangement according to the invention is that it allows a non-acoustic voice link within the shielded room without the use of cables, specifically by transmitting audio signals by radio—also referred to for short in the following text as voice radio signals. One major problem in the examination and/or treatment of a human or animal body relates to the communication between the personnel, that is to say for example between the doctor and the medically technical personnel, and/or the personnel and a patient who is to be examined or to be treated. The communication within the shielded room is made more difficult by the normally large amount of noise developed by the gradient coils of the magnetic resonance imaging device while images are being recorded. This is the purpose of the invention, in that the invention makes it possible to use radio signals for transmission of acoustic signals, that is to say for example for transmission of voice signals, within the shielded room.

One preferred refinement of the invention provides that the radio link between the at least one antenna which is connected to the at least one further communication device and the at least one communication device which is arranged within the shielded room is in a frequency range below 10 MHz, and the at least one communication device has two hearing protection capsules and an optical waveguide, which transmits a received audio signal optically from the one hearing protection capsule to the other hearing protection capsule. This refinement makes use of a surprising synergy effect which the inventors have found: the frequency range below 10 MHz is free of harmonics which the magnetic field of the magnetic resonance imaging device produces, thus making it possible to transmit voice signals largely without interference. The optical transmission of the voice signals from one hearing protection capsule to the other hearing protection capsule makes it possible to avoid electrical connecting conductors between the hearing protection capsules: because of the anatomy, an electrical connecting conductor between the hearing protection capsules would normally have a length of about 20 cm, thus resulting in an interfering antenna being formed for the working frequency range of the magnetic resonance imaging device and, where possible, a shielding effect could occur for the radio frequency range below 10 MHz. The optical transmission of the voice signals effectively prevents the formation of an antenna, however, and shielding by the electrical connecting conductors which would otherwise be required. In other words, the combination of hearing protection capsule connection without any conductors or antennas, with voice signal transmission in a frequency range below 10 MHz, preferably in a frequency range between 1 MHz and 10 MHz, allows low-interference magnetic resonance imaging and, at the same time, voice transmission with little interference.

Preferably, the at least one further communication device is an external communication device which is located outside the shielded room. Preferably, the at least one communication device is connected by a voice radio link to the at least one further communication device.

According to another preferred refinement of the arrangement, the electro-acoustic transducer is physically arranged in a hearing protection means or is integrated therein. Such integration allows the personnel and/or the patient to be protected in a simple manner against the noise from the gradient coils of the magnetic resonance imaging device, while at the same time allowing communication between the personnel and/or with the patient.

In order to prevent, or at least reduce as far as possible, any interference with the magnetic resonance imaging device caused by the radio signals and/or interference for the radio signals caused by the magnetic resonance imaging device, it is considered to be advantageous for the radio signals to be transmitted in a frequency range below 10 MHz (preferably in a frequency range between 1 MHz and 10 MHz), and/or in a frequency range above 300 MHz. The radio or voice radio signals can be transmitted in analog form, preferably by amplitude modulation or frequency modulation. However, it is considered to be preferable for the radio or voice radio signals to be transmitted in a digitally coded form, preferably with amplitude modulation or frequency modulation.

Particularly preferably, radio transmission between communication devices which are arranged within the shielded room and/or radio transmission between communication devices which are arranged within the shielded room and those communication devices which are arranged outside the shielded room takes place in the frequency range between 2.4 GHz and 2.5 GHz, for example in accordance with or based on the Bluetooth standard in accordance with IEEE 802.15.1 or in accordance with or based on the WLAN (Wireless Local Area Network) standard in accordance with IEEE 802.11. Alternatively or additionally, the radio transmission can take place in the frequency range between 5 GHz and 6 GHz, for example in accordance with or based on the WLAN standard.

In order to achieve particularly good MRI (magnetic resonance imaging device) compatibility for the electro-acoustic transducer and particularly good MRI compatibility of the communication device overall, it is considered to be advantageous for the communication device and the electro-acoustic transducer to have as little ferromagnetic material as possible, or no ferromagnetic material, that is to say for example little or no iron. Ferromagnetic material within the shielded room would be subject to very large acceleration forces in the magnetic field produced by the magnetic resonance imaging device, depending on the distance therefrom, thus making it possible for a hazard to occur to personnel and/or equipment, such as the magnetic resonance imaging device for example; in addition ferromagnetic material can adversely affect the image quality of the magnetic resonance imaging device.

With a view to the magnetic resonance imaging device causing little interference to the electro-acoustic transducer and, conversely, with a view to the electro-acoustic transducer causing little interference to the magnetic resonance imaging device, it is considered to be particularly advantageous for the electro-acoustic transducer to operate capacitively. For example, the electro-acoustic transducer may comprise a piezoelectric element, or may be formed by such an element. In a refinement such as this it is advantageously possible to completely dispense with, or virtually completely dispense with, electromagnetic conversion and the use of ferromagnetic material.

In order to provide a microphone function, the communication device may also have an acousto-electrical transducer, which is suitable for conversion of an acoustic signal, received within the shielded room, to an electrical audio signal. In a refinement such as this, the communication device is preferably also suitable for using the electrical audio signal to form a radio signal, and for transmitting this. For the reasons mentioned above, an acousto-electrical transducer such as this is preferably produced with as little ferromagnetic material as possible, or even with no ferromagnetic material.

Preferably, the acousto-electrical transducer has an acousto-optical transducer unit and an optoelectrical transducer unit which is arranged downstream from the acousto-optical transducer unit, with the acousto-optical transducer unit being suitable for conversion of the acoustic signal received within the shielded room to an optical signal, and with the optoelectrical transducer unit being suitable for conversion of the optical signal from the acousto-optical transducer unit to an electrical audio signal. A refinement of the acousto-electrical transducer such as this allows production without ferromagnetic material, or at least with little ferromagnetic material, in a particularly simple and cost-effective manner, nevertheless with a good transducer characteristic.

In this case, acousto-optical conversion means conversion of an acoustic signal to an optical signal, optoacoustic conversion means conversion of an optical signal to an acoustic signal, electro-acoustic conversion means conversion of an electrical signal to an acoustic signal, and acousto-electrical conversion means conversion of an acoustic signal to an electrical signal.

By way of example, the communication device may be connected to an external communication device which is located outside the shielded room. Alternatively or additionally, the communication device may be connected to at least one other communication device, which is located within the shielded room.

The invention also relates to a communication device for an arrangement as described above.

With regard to a communication device such as this, the invention provides that this communication device has a radio receiving device for receiving a radio signal as well as a piezo-electric element which is connected thereto and is suitable for conversion of an audio signal transmitted by the radio signal to an acoustic signal.

With regard to the advantages of the communication device according to the invention, reference is made to the advantages of the arrangement according to the invention as described above, since the advantages of the communication device according to the invention correspond essentially to those of the arrangement according to the invention.

The invention also relates to a hearing protection means for an arrangement as described above.

With respect to such hearing protection means, the invention provides that this hearing protection means has a radio receiving device for reception of a radio signal as well as a piezoelectric element which is connected thereto and is suitable for conversion of an audio signal transmitted by the radio signal to an acoustic signal.

With respect to the advantages of the hearing protection means according to the invention, reference is made to the advantages of the arrangement according to the invention as described above, since the advantages of the hearing protection means according to the invention correspond essentially to those of the arrangement according to the invention.

Preferably, the hearing protection means has an electro-optical transducer and an optoelectrical transducer, with the function of the electro-optical transducer being to convert an electrical audio signal to an optical audio signal, and to transmit this optical audio signal via an optical waveguide from the one hearing protection capsule to the other hearing protection capsule. The optical waveguide is preferably integrated in the headpiece of the hearing protection means or is connected thereto.

The invention will be explained in more detail in the following text with reference to exemplary embodiments; in this case, by way of example:

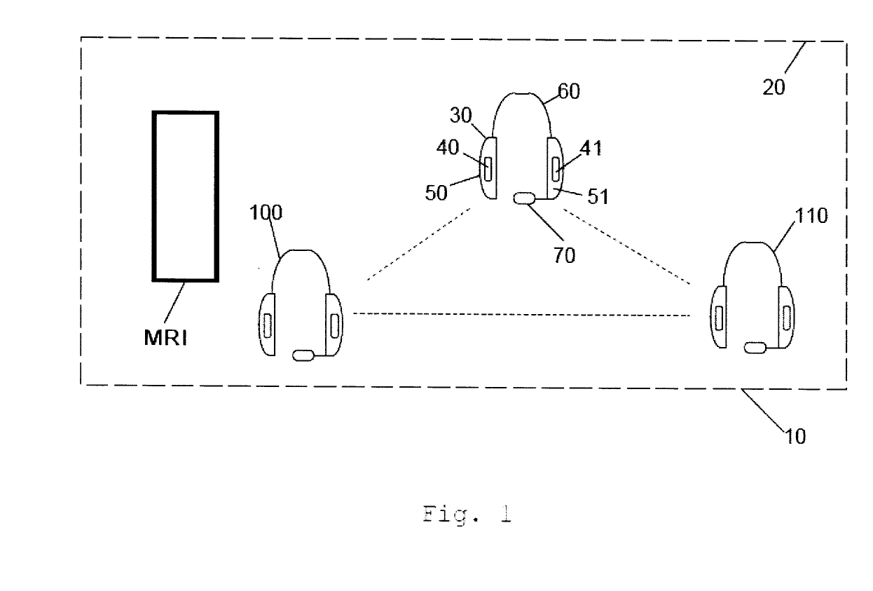

FIG. 1 shows a first exemplary embodiment of an arrangement according to the invention for examination and/or treatment of a human or animal body, with the arrangement comprising a hearing protection means with a communication device,

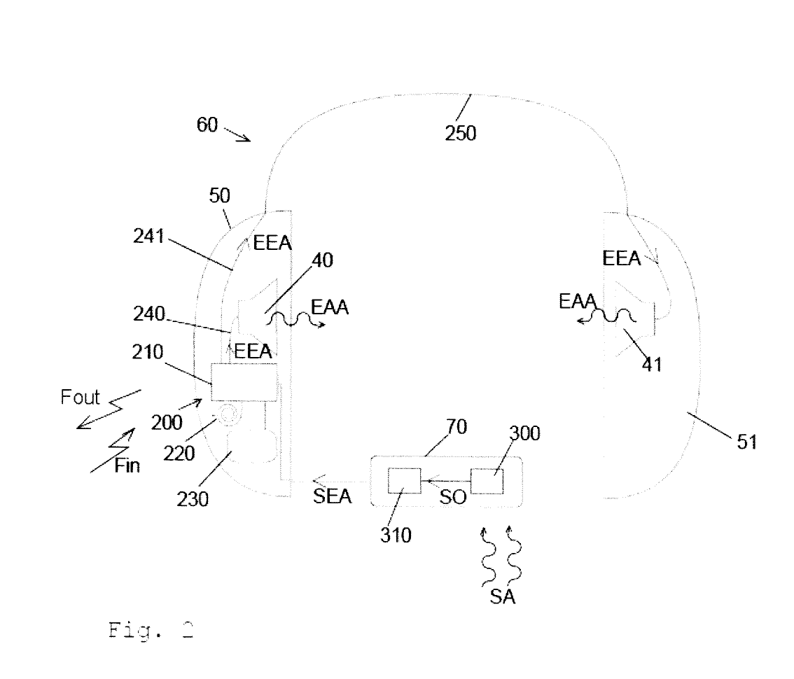

FIG. 2 shows one exemplary embodiment of the hearing protection means shown in FIG. 1,

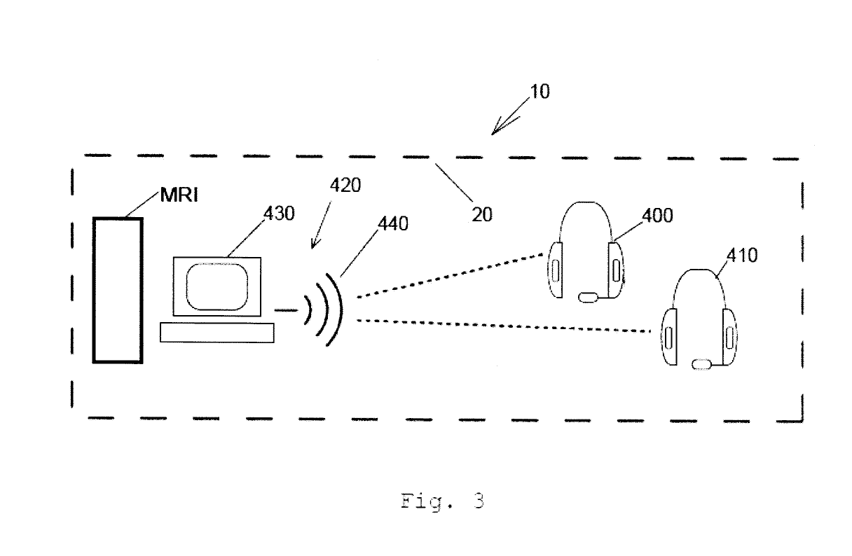

FIG. 3 shows a second exemplary embodiment of an arrangement according to the invention for examination and/or treatment of a human or animal body, with different types of communication devices being linked by radio within a shielded room,

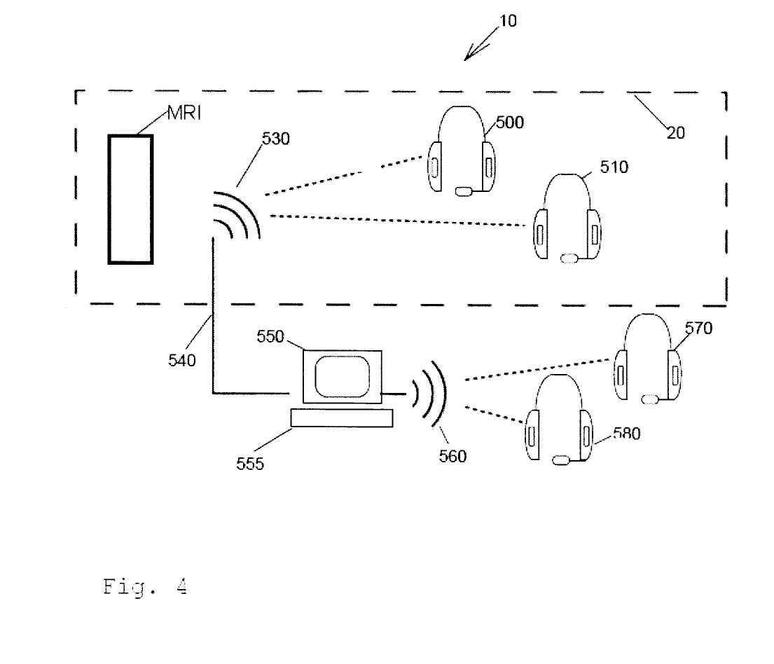

FIG. 4 shows a third exemplary embodiment of an arrangement according to the invention for examination and/or treatment of a human or animal body, with communication devices which are arranged within a shielded room being linked by radio to an external communication device which is located outside the shielded room, and

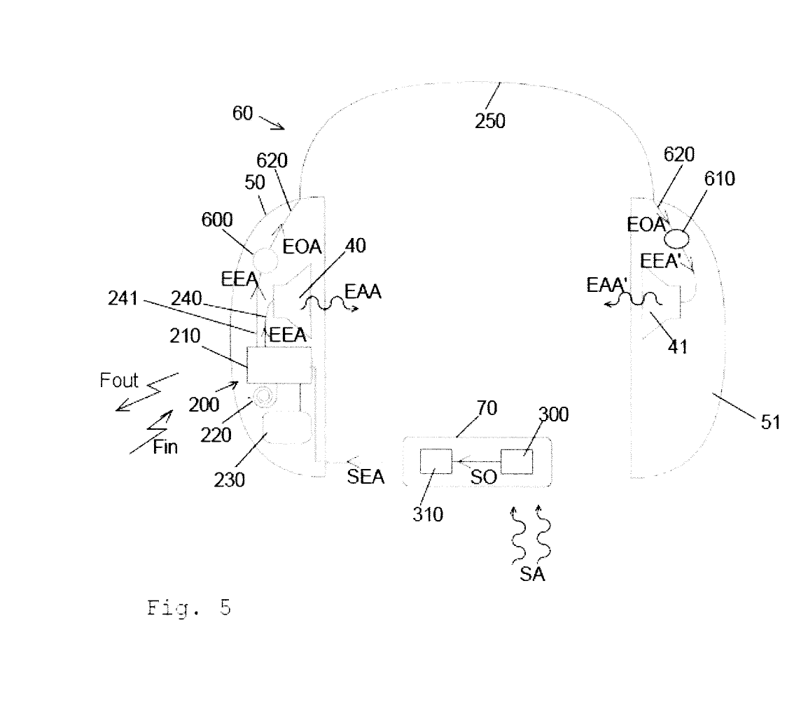

FIG. 5 shows a further exemplary embodiment of a hearing protection means according to the invention, which can be used in one of the arrangements shown in FIGS. 1 and 3 to 4 and has an optical signal link between the hearing protection capsules.

For the sake of clarity, the same reference symbols are always used for identical or comparable components in the figures.

FIG. 1 shows an arrangement 10 for examination and/or treatment of a human or animal body, in the form of a schematic illustration. The figure shows an electromagnetically shielded room 20, which forms an electromagnetically closed, or at least approximately electromagnetically closed Faraday cage. A magnetic resonance imaging device is located within the shielded room 20 and is annotated with the reference symbol MRI.

FIG. 1 furthermore shows a communication device 30 which comprises two electro-acoustic transducers 40 and 41. The two electro-acoustic transducers 40 and 41 are integrated in hearing protection capsules 50 and 51 of a hearing protection means 60, and form loudspeakers within the hearing protection means 60.

Furthermore, FIG. 1 shows that the communication device 30 and therefore the hearing protection means 60 may also have an acousto-electrical transducer 70. The acousto-electrical transducer 70 forms a microphone, which is arranged outside the hearing protection capsules 50 and 51 of the hearing protection means 60.

The communication device 30 is suitable for receiving radio signals and for converting them with the aid of the two electro-acoustic transducers 40 and 41 in the hearing protection capsules 50 and 51 to acoustic signals, in order to feed these to the auditory channels of a person who is wearing the hearing protection means 60. Furthermore, the communication device 30 is suitable for conversion of acoustic signals, which originate by way of example from the person who is wearing the hearing protection means, to an electrical audio signal with the aid of the acousto-electrical transducer 70, and for transmitting this audio signal as a radio signal.

As can be seen from FIG. 1, the communication device 30 can be used to communicate with other communication devices 100 and 110. The two communication devices 100 and 110 may, for example, by physically the same as the communication device 30, that is to say they may each form a corresponding hearing protection means as well.

The arrangement illustrated in FIG. 1 makes it possible for three people who are located within the shielded room 20 to communicate with one another by radio, and at the same time to be protected against the noise of the magnetic resonance imaging device.

FIG. 2 shows one exemplary embodiment of the communication device 30 and the hearing protection means 60 shown in FIG. 1. As can be seen, the communication device 30 has a radio receiving device 200, which comprises a control device 210, an antenna 220 and a battery 230. The control device 210 is connected to the two electro-acoustic transducers 40 and 41, for example by two electrical cables, which are annotated with the reference symbols 240 and 241 in FIG. 2.

FIG. 2 furthermore shows the acousto-electrical transducer 70 in detail. As can be seen, the acousto-electrical transducer 70 may comprise an acousto-optical transducer unit 300 and an optoelectrical transducer unit 310, which is arranged downstream from the acousto-optical transducer unit 300. The optoelectrical transducer unit 310 is connected to the control device 210.

By way of example, the control device 210 may be formed by an electrical board which may have an A/D and/or D/A converter, a coding device, a modulation and/or demodulation device, a transceiver and/or a transmitter.

The electrical line 241 which connects the control device 210 to the electro-acoustic transducer 41 in the hearing protection capsule 51 is, for example, integrated in a headpiece 250 of the hearing protection means 60, or is connected thereto.

The hearing protection means 60 and the communication device 30 may, for example, be operated as follows:

An acoustic signal SA which is received within a shielded room is received by the acousto-optical transducer unit 300 of the acousto-electrical transducer 70 and is converted to an optical signal SO. The optical signal SO is passed to the optoelectrical transducer unit 310, which uses the optical signal SO to form an electrical audio signal SEA to be transmitted, and feeds this into the control device 210. The control device 210 together with the antenna 220 and with the electrical audio signal SEA forms a radio signal Fout, which is transmitted by the antenna 220 and is sent, for example, to the two communication devices 100 and 110 shown in FIG. 1.

The hearing protection means 60 and the communication device 30 can be used in a corresponding manner to receive a radio signal Fin. A radio signal Fin such as this is received by the antenna 220 and is evaluated by the control device 210. An electrical audio signal EEA which is transmitted in the radio signal Fin is extracted from the radio signal Fin and is transmitted to the two electro-acoustic transducers 40 and 41. The two electro-acoustic transducers 40 and 41 use the received electrical audio signal EEA to form an acoustic signal EAA, and feed this indirectly or directly into the ears of a person who is wearing the hearing protection means 60.

In order to prevent the communication device 30 from interfering with the work of the magnetic resonance imaging device in the shielded room, and/or to prevent the operation of the communication device 30 from being interfered with by the magnetic resonance imaging device, the acousto-electrical transducer 70 and the two electro-acoustic transducers 40 and 41 are as free of ferromagnetic material as possible, and in particular are as free of iron as possible. The operation of the two electro-acoustic transducers 40 and 41 is therefore preferably not based on an electromagnetic effect, but on a capacitive effect. By way of example, the two electro-acoustic transducers 40 and 41 each comprise one or more piezoelectric elements. In other words, the electrical audio signals EEA are converted to acoustic signals EAA by the two electro-acoustic transducers 40 and 41, preferably on the basis of a capacitive effect, in particular a piezoelectric effect.

In a corresponding manner, the acousto-electrical transducer 70 preferably has little ferromagnetic material, or is free of ferromagnetic material, and in particular is formed without an electromagnetic transducer unit. In the exemplary embodiment shown in FIG. 2, the acousto-electrical transducer 70 has two different transducer units, specifically the acousto-optical transducer unit 300 and the downstream optoelectrical transducer unit 310; both transducer units can operate without using an electromagnetic effect.

By way of example, the acousto-optical transducer unit 300 may comprise a mirror element which is coupled to a membrane which can oscillate. When an acoustic wave strikes the membrane, the membrane will oscillate and will correspondingly deflect the mirror element. Incident light can thus be modulated, forming the optical signal SO.

The optoelectrical transducer unit 310 may be formed, for example, by a photodetector, for example a semiconductor photodetector.

FIG. 3 shows an arrangement 10 for examination and/or treatment of a human or animal body, in which three communication devices 400, 410 and 420 are located within a shielded room 20.

The two communication devices 400 and 410 are each formed, for example, by a hearing protection means 60, as is illustrated by way of example in FIG. 2.

By way of example, the communication device 420 comprises a computer 430, which is connected via an antenna 440 to the two communication devices 400 and 410.

FIG. 4 shows an exemplary embodiment of an arrangement 10 for examination and/or treatment of a human or animal body, in which two communication devices 500 and 510 are arranged within the shielded room 20 of the arrangement 10. The two communication devices 500 and 510 are connected to one another and to an antenna 530, which is connected via an interface, for example in the form of a cable link 540, to an external communication device 550.

By way of example, the external communication device 550 may have a computer 555 and a further antenna 560, via which the computer 555, and therefore indirectly the two communication devices 500 and 510 as well, can be connected to further external communication devices 570 and 580.

FIG. 5 shows a further exemplary embodiment of the communication device 30 and the hearing protection means 60 shown in FIG. 1. As can be seen, the communication device 30 as shown in FIG. 5 has an electro-optical transducer 600 and an optoelectrical transducer 610. The function of the electro-optical transducer 600 is to convert the received electrical audio signal EEA to a received optical audio signal EOA, and to transmit this optical signal EOA via an optical waveguide 620 from the left-hand hearing protection capsule 50 in FIG. 1 to the right-hand hearing protection capsule 51 in FIG. 5.

The optical waveguide 620 is integrated in the headpiece 250 of the hearing protection means 60, or is connected thereto, and is connected to the optoelectrical transducer 610, which uses the received optical audio signal EOA to form a received electrical audio signal EEA′, and feeds this into the electro-acoustic transducer 41 in the right-hand hearing protection capsule 51, in order to form the acoustic output signal EAA′.

Apart from this, the communication device 30 and the hearing protection means 60 as shown in FIG. 5 correspond respectively to the communication device 30 and the hearing protection means 60 shown in FIG. 2.

### LIST OF REFERENCE SYMBOLS

10 Arrangement

20 Shielded room

30 Communication device

40 Transducer

41 Transducer

50 Hearing protection capsule

51 Hearing protection capsule

60 Hearing protection means

70 Transducer

100 Communication device

110 Communication device

200 Radio receiving device

210 Control device

220 Antenna

230 Battery

240 Electrical cable

241 Electrical cable

250 Headpiece

300 Acousto-optical transducer unit

310 Optoelectrical transducer unit

400 Communication device

410 Communication device

420 Communication device

430 Computer

440 Antenna

500 Communication device

510 Communication device

530 Antenna

540 Cable link

550 Communication device

555 Computer

560 Antenna

570 Communication device

580 Communication device

600 Electro-optical transducer

610 Optoelectrical transducer

620 Optical waveguide

EAA Acoustic audio signal

EAA′ Acoustic audio signal

EEA Electrical audio signal

EEA′ Electrical audio signal

EOA Optical audio signal

Fout Radio signal

Fin Radio signal

MRI Magnetic resonance imaging device

SA Acoustic audio signal

SEA Electrical audio signal

SO Optical audio signal

1. An arrangement (10) for examination and/or treatment of a human or animal body having
a shielded room (20),
a magnetic resonance imaging device (MRI) which is arranged within the shielded room, and
at least one communication device (30), which is arranged within the shielded room and is suitable for receiving a radio signal for transmission of a voice signal, and has at least one electro-acoustic transducer (40, 41) which is suitable for conversion of an audio signal, which has been transmitted by the radio signal (Fin), to an acoustic signal (EAA),
wherein at least one antenna, which is connected to at least one further communication device, is arranged in the shielded room and is connected by radio to the at least one communication device (30) which is arranged within the shielded room.

2. The arrangement as claimed in claim 1,
wherein the radio link between the at least one antenna which is connected to the at least one further communication device and the at least one communication device (30) which is arranged within the shielded room is a voice radio link and is in a frequency range below 10 MHz, and
the at least one communication device has two hearing protection capsules and an optical waveguide (620), which transmits a received audio signal optically from the one hearing protection capsule (50) to the other hearing protection capsule (51).

3. The arrangement as claimed in claim 2, characterized in that
wherein the at least one further communication device is an external communication device which is located outside the shielded room, and - the at least one communication device is connected by a voice radio link to the at least one further communication device.

4. The arrangement as claimed in claim 1, wherein the at least one electro-acoustic transducer comprises a capacitively operating element for electro-acoustic conversion, or is formed by such an element.

5. The arrangement as claimed in claim 1, wherein the electro-acoustic transducer comprises a piezoelectric element for electro-acoustic conversion, or is formed by such an element.

6. The arrangement as claimed in claim 1, wherein the electro-acoustic transducer is integrated in a hearing protection means (60).

7. The arrangement as claimed in claim 1,
wherein the at least one communication device has an acousto-electrical transducer (70), which is suitable for conversion of an acoustic signal (SA), which is received within the shielded room, to an electrical audio signal (SEA), and
the at least one communication device is suitable for forming a radio signal (Fout) using the electrical audio signal, and for transmission thereof.

8. The arrangement as claimed in claim 7,
wherein the acousto-electrical transducer has an acousto-optical transducer unit (300) and an optoelectrical transducer unit (310) which is arranged downstream from the acousto-optical transducer unit,
with the acousto-optical transducer unit being suitable for conversion of the acoustic signal received within the shielded room to an optical signal (SO), and
with the optoelectrical transducer unit being suitable for conversion of the optical signal from the acousto-optical transducer unit to the electrical audio signal.

9. The arrangement as claimed in claim 1, wherein the at least one further communication device is an external communication device, which is located outside the shielded room.

10. The arrangement as claimed in claim 1, wherein the at least one further communication device is located within the shielded room.

11. The arrangement as claimed in claim 1, wherein the radio link between the at least one antenna which is connected to the at least one further communication device and the at least one communication device (30) which is arranged within the shielded room is a voice radio link, and is in a frequency range below 10 MHz and/or above 300 MHz.

12. The arrangement as claimed claim 1, wherein the at least one communication device has two hearing protection capsules and an optical waveguide (620), which transmits a received audio signal optically from the one hearing protection capsule (50) to the other hearing protection capsule (51).

13. A hearing protection means for an arrangement as claimed in claim 1,
wherein the hearing protection means (60) has a radio receiving device (200) for reception of a voice radio signal,
with the radio receiving device being suitable for operation of a voice radio link in a frequency range below 10 MHz.

14. The hearing protection means as claimed in claim 13, wherein the hearing protection means (60) has two hearing protection capsules and an optical waveguide (620), which transmits a received audio signal optically from the one hearing protection capsule (50) to the other hearing protection capsule (51).

15. A hearing protection means for an arrangement as claimed in claim 1, wherein the hearing protection means (60) has two hearing protection capsules and an optical waveguide (620), which transmits a received audio signal optically from the one hearing protection capsule (50) to the other hearing protection capsule (51).

16. A communication device for an arrangement as claimed claim 1, wherein the communication device has a radio receiving device (200) for reception of radio signals as well as a piezoelectric element which is connected thereto and is suitable for conversion of an audio signal transmitted by the radio signal to an acoustic signal.

17. A hearing protection means for an arrangement as claimed claim 1, wherein the hearing protection means (60) has a radio receiving device (200) for reception of radio signals as well as a piezoelectric element which is connected thereto and is suitable for conversion of an audio signal transmitted by the radio signal to an acoustic signal.
